(19) United States
(12) Patent Application Publication
(10) Pub. No.: US 2015/0291628 A1
(43) Pub. Date: Oct. 15, 2015

(54) NOVEL AZA BODIPY COMPOUND FOR THE SELECTIVE DETECTION OF NITRITE IONS IN WATER AND A PROCESS FOR PREPARATION THEREOF

(71) Applicant: COUNCIL OF SCIENTIFIC & INDUSTRIAL RESEARCH, New Delhi (IN)

(72) Inventors: Ramaiah Danaboyina, Thiruvananthapuram (IN); Adarsh Nagappanpillai, Thiruwananthapuram (IN); Shanmugasundaram Madhesh, Thiruwananthapuram (IN)

(21) Appl. No.: 14/439,049

(22) PCT Filed: Jan. 28, 2014
(86) PCT No.: PCT/IN2014/000067

§ 371 Date: Apr. 28, 2015

(30) Foreign Application Priority Data
Jan. 28, 2013 (IN) 206/DEL/2013

(51) Int. Cl. C07F 5/02; G01N 33/18; G01N 21/78

(52) U.S. Cl. CPC C07F 5/022; G01N 21/78; G01N 33/188

(57) ABSTRACT

The present invention provides aza-BODIPY compound having formula 1 useful for detection of nitrite ions in an aqueous medium. The invention also provides a process for the preparation of aza-BODIPY compound having formula 1. For detecting nitrite ions, a dipstick device made by coating the assay powder of formula 1 in alumina over a thermoplastic or a glass solid support is used. The detection event can be monitored by noting the color change on the surface of the dipstick. Detection event by means of color change is selective for nitrite ions when compared to all other biologically important ions like SO₄²⁻, Cl⁻, HSO₃⁻, CO₃²⁻, CH₃COO⁻, NO₃⁻, S₂O₃²⁻, N₃⁻.

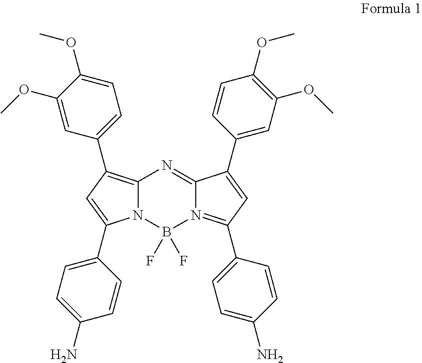

### FIELD OF THE INVENTION

The present invention relates to a novel aza BODIPY (4,4-difluoro-4-bora-3a,4a-diaza-s-indacenes) compound of formula 1. The present invention also relates to a process for the preparation of aza-BODIPY dye. The present invention particularly relates to formulation of a colorimetric nitrite sensor and demonstration of its application as a sensor for the selective and sensitive detection of nitrite ions in the aqueous medium. More particularly the present invention relates to on the spot selective detection of nitrite ions (NO₂⁻) from the water samples containing different competitive anions.

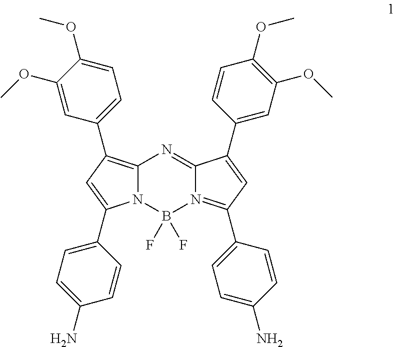

The present invention also provides a process for the preparation of aza-BODIPY dye of formula 1 and the formulation of a colorimetric nitrite sensor. The aza-BODIPY dye can be coated over a plastic/glass/paper back support for the fabrication of dipstick devices. Thereby prepared device in particular can be used for on the spot selective detection of nitrite ions (NO₂⁻) from the water samples containing different competitive anions.

### BACKGROUND OF THE INVENTION

Nitrite (NO₂⁻) ion contamination of rural drinking water supplies by livestock waste, organic wastes and chemical fertilizers continues to be a problem throughout the world. Nitrite (NO₂⁻) is among the three inorganic nitrogen containing ions (NH₄⁺, NO₂⁻, and NO₃⁻) in the environment which are essential nutrients for the growth of plants. Meanwhile, it is of major concern to scientists owing to its adverse effects to animal and human health during the chronic exposure at trace levels or negligent intake of high levels. One study estimates that greater than million Americans are using self-supplied water with nitrate concentrations exceeding the maximum contaminant level (MCL) defined by the Environmental Protection Agency (EPA). Nitrites are usually adopted as a food preservative to extend the shelf life of processed meat, however, they are also acknowledged to be important precursors upon interaction with proteins for producing carcinogenic N-nitrosamines, which are extremely detrimental to human health. Water is another major accessible source through which the safety of human health is threatened by dangerous levels of nitrite (or nitrate). They are commonly found in ground water than in surface water, and the principal contributors are livestock manure, chemical fertilizers, and erosion of natural deposits. A number of medical issues are believed to be associated with nitrite (or nitrate) intake. One typical example is the infant methemoglobinemia, also known as “blue baby syndrome”, which is characterized by bluish coloration of the skin. In view of the toxicity of nitrite, the U.S. Environmental Protection Agency (EPA) defined the maximum contamination level (MCL) of nitrite in drinking water to be 1 ppm (21.7 μM) and the analogous guideline value set by the World Health Organization (WHO) is 3 ppm. References may be made to M. J. Hill, Nitrates and Nitrites in Food and Water, 1^{st} Ed., Woodhead Publishing Limited, England, 1996; U.S. patent Ser. No. 00/577,6715 A, 1998; U.S. patent Ser. No. 00/765,5473 B2, 2010; U.S. Pat. No. 3,802,842, 1974; B. Liang, M. Iwatsuki, T. Fukasawa, The Analyst, 1994, 119, 2113-2117; Z. Xue, Z. Wu, S. Han, Anal. Methods, 2012, 4, 2021-2026; M. C. Archer, S. D. Clark, J. E. Thilly, S. R. Tannenbaum, J. Am. Chem. Soc. 2009, 131, 6362-6363; United States Environmental Protection Agency. National Primary Drinking Water Regulations: Contaminant Specific Fact Sheets, Inorganic Chemicals, consumer version; Washington, D.C., 2009; World Health Organization. Guidelines for Drinking Water Quality: incorporating 1st and 2 nd addenda, Vol. 1, Recommendations.—3^{rd} Ed., WHO Press, Geneva, Switzerland, 2008.

Nitrite is known to be chemically unstable in the environment. Owing to this reactivity, a rapid detection procedure is preferable, especially for on-site analysis. Various techniques that have been developed for the detection of nitrite ions include the spectroscopy (photometry and fluorometry), electrochemistry, and chemiluminescence. The spectrophotometric method is generally simple and selective, comprises of the nitrous acid specific diazotization of aromatic amine and subsequent coupling reaction with suitable aromatic reagent to yield a highly colored azo dye, the intensity of which is related to the original nitrite concentration. This method originates from the classic Griess reaction. The Griess test, proceeds by nitrite dependent diazotization of sulfanilamide under acidic conditions and coupling of the in situ generated diazonium ion with N-(1-naphthyl)-ethylenediamine to afford azochromophores. One of the major disadvantages of Griess reagent is the use of high concentrations of hazardous reagents. In addition to the environmental concern, solution pH and temperature for the reaction also needs particular attention, thereby complicating the detection procedure. Electrochemical methods and chemiluminescence are another two methodologies for the determination of nitrite, but both of them need to be performed on expensive instruments, limiting their widespread applications in real samples. References may be made to M. J. Moorcroft, J. Davis, R. G. Compton, Talanta, 2001, 54, 785-803; N. Wang, X. Cao, X. Cai, Y. Xu, L. Guo, Analyst, 2010, 135, 2106-2110; W. Ko, W. Chen, C. Cheng, K. Lin, Sens. Actuators B, 2009, 137, 437-441; Z. Lin, W. Xue, H. Chen, J. Lin, Anal. Chem., 2011, 83, 8245-8251; J. Li, Q. Li, C. Lu, L. Zhao, D. Ye, L. Luo, Y. Ding, Q. Chen, X. Liu, Analyst, 2011, 136, 4563-4569.

In this regard development of a single molecule which can act as an efficient nitrite sensor in aqueous media as well as in solid state devices is a challenging task. The boron complexes 4,4-difluoro-4-bora-3a,4a-diaza-s-indacenes, abbreviated as BODIPYs hold great promise as ideal sensitizers owing to their favourable properties. These systems exhibit strong absorption in the range 500-600 nm and significant fluorescence quantum yields and high photostability. In contrast, the aza-dipyrromethenes and their boron complexes (aza-BODIPYs) show around 100 nm bathochromic shifted absoprtion when compared to BODIPYs attracted less attention and have been studied only in the last decade. References may be made to S. Ozlem, E. U. Akkaya, J. Am. Chem. Soc. 2009, 131, 48-49; T. Yogo, Y. Urano, Y. Ishitsuka, F. Maniwa, T. Nagano, J. Am. Chem. Soc. 2005, 127, 12162-12163; A. Gorman, J. Killoran, C. O'Shea, T. Kenna, W. M. Gallagher, D. F. O'Shea, J. Am. Chem. Soc. 2004, 126, 10619-10631; A. Loudet, K. Burgess, Chem. Rev. 2007, 107, 4891-4932; R. Ziessel, B. D. Allen, D. B. Rewinska, A. Harriman, Chem. Eur. J., 2009, 15, 7382-7393; N. Adarsh, R. R. Avirah, D. Ramaiah, Org. Lett. 2010, 12, 5720-5723.

The main disadvantages of the existing molecular probes for the practical application of the detection of nitrite ions in aqueous media are:
1. The first disadvantage is that the detection still requires precise transfer of solutions, usage of sophisticated instrumental techniques etc., making it more useful to people who do not have any scientific background.
2. Second disadvantage is the low sensitivity and selectivity of the probes towards nitrite ions for an instrument free observation.
3. Third disadvantage is the inability of the fabrication of simple devices like dipsticks, strips etc. out of these probes by which the detection can be made much simpler and convenient.
4. The fourth and significant disadvantage is the lack of single and simple molecule as a probe for nitrite ions, the Griess reagent, most common nitrite sensor which is in commercial use is a mixture of three components.

In the present invention, we have developed an aza-BODIPY based nitrite sensor which is showing good selectivity for the nitrite ions over a wide range of competitive anions. Also the sensor showed much improved sensitivity with a limit of detection value of 300-500 nM as compared to the classical nitrite sensor Griess reagent (LOD=1 μM). The quantitative estimation of nitrite ions in a given sample also can be done with an allowed error limit. To avoid the use of sophisticated instruments for the detection technique, we formulated a simple and efficient dipstick device for on the spot sensing of nitrite ions.

### OBJECTIVES OF THE INVENTION

The main object of the present invention is to provide a single molecule aza-BODIPY dye of formula 1 useful for selective detection of nitrite ions in solution.

Another object of the present invention is to provide a process for the preparation of aza-BODIPY dyes of formula 1.

Still another object of the present invention is to provide a technique for detecting nitrite ions in a relatively simple and convenient manner using aza-BODIPY dyes of formula 1, specifically suited for practical application of molecular probes such as sensors for nitrite ions.

Yet another object of the present invention is to provide a simple and accurate method for quantitative estimation of nitrite ions in a given sample.

Still another object of the present invention is to provide a simple and efficient dipstick device for selective detection of nitrite ions using an aza-BODIPY of formula 1.

Yet another object of the present invention is to provide an accurate, fast, real time method for detection nitrite ions in aqueous samples.

### SUMMARY OF THE INVENTION

Accordingly, the present invention provides an aza-BODIPY compound of formula 1 and its salt thereof useful for selective detection of nitrite ions in a solution.

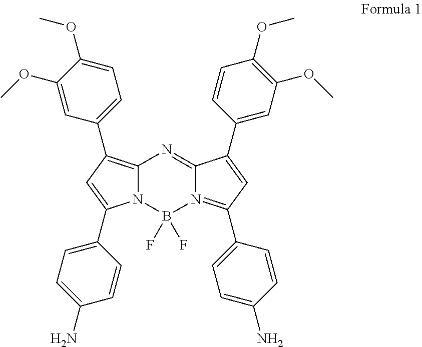

In an embodiment of the present invention, the compound shows high thermal stability up to a temperature of 280° C.

In an embodiment of the present invention, the compound is useful for detection of nitrite ions in an aqueous medium.

An embodiment of the present invention provides a process for the preparation of an aza-BODIPY compound of formula 1, the process comprising:
a) adding 4-aminoacetophenone to a solution of NaOH and stirring for 10 minutes to obtain a reaction mixture;
b) adding 3,4-dimethoxy benzaldehyde dropwise to the reaction mixture and stirring for 6 hours to obtain a precipitate;
c) filtering the precipitate and washing with water to obtain chalcone;
d) reacting the chalcone in ethanol with nitromethane in presence of activated K₂CO₃ and refluxing for a time period of 24 hours to obtain a mixture;
e) washing the mixture with water and extracting with chloroform;
f) removing the solvent to obtain a residue and separating by column chromatography to obtain nitromethane adduct;
g) reacting the nitromethane adduct with ammonium acetate in ethanol and heating under reflux for a time period of 48 hours to obtain a product;
h) filtering the product and washing with ethanol, drying and recrystallizing from chloroform to obtain azadipyrromethene as greenish crystals with a metallic luster;
i) reacting azadipyrromethene in dry dichloromethane with boron trifluoride diethyl etherate in presence of diisopropylethylamine (DIEA) and stirring for a time period of 15 hours at a temperature of 25° C.;
j) evaporating the solvent and washing the residue obtained with water followed by extracting with chloroform; and
k) separating by column chromatography to obtain aza-BODIPY as a purple colored solid.

In an embodiment of the present invention the compound exhibits a highly intense purple color having strong NIR absorption in the range of 600-850 nm with an absorption maximum at 750 nm.

In an embodiment of the present invention, the compound exhibits NIR emission in methanol in the range of 700-900 nm with an emission maximum at about 825 nm.

In an embodiment of the present invention, the compound exhibits strong absorption with an extinction coefficient of 4.8±0.2×10⁴ M⁻¹ cm⁻¹.

In an embodiment of the present invention the compound can be protonated by adding dilute acids, preferably HCl.

In an embodiment of the present invention, the protonated form of the compound has intense blue color with a broad absorption band in the range of 500-800 nm, with a maximum of 570 nm.

In an embodiment of the present invention, the protonated form of the compound is useful for detection of NO₂⁻ ions in presence of other anions selected from the group consisting of SO₄²⁻, Cl⁻, HSO₃⁻, CO₃²⁻, CH₃COO⁻, NO₃, S₂O₃²⁻ and N₃⁻ in solution form.

In an embodiment of the present invention, the compound shows the limit of detection (LOD) of 500 nM in aqueous medium.

An embodiment of the present invention provides a dipstick device useful for selective detection of NO₂⁻ ions in water using the compound of the present invention, wherein the device comprises the compound of Formula 1 fixed on the surface of a support selected from the group consisting of plastic, glass and paper backing.

Another embodiment of the present invention provides a process for preparing a dipstick device useful for selective detection of NO₂⁻ ions in water using the compound of formula 1 comprising the steps:
dissolving compound of formula 1 in dichloromethane to obtain a mixture;
adding finely powdered silica or alumina and allowing the dichloromethane to evaporate after leaving for 10 minutes to obtain an assay;
fixing the assay over the surface of a support to obtain a dipstick; and exposing the dipstick to HCl gas followed by dipping it in an sample to detect nitrite ions.

An embodiment of the present invention provides a process for preparing a dipstick device wherein the support is made of plastic, glass or paper backing selected from the group consisting of thermoplastic, absorption pads, glass rods and paper strips.

An embodiment of the present invention provides a process for preparing a dipstick device useful for testing of analytical sample containing nitrite ion concentration as low as 2.0±0.5×10⁻⁵ M.

### BRIEF DESCRIPTION OF THE DRAWINGS

FIG. 1 The aza-BODIPY compound of formula 1.

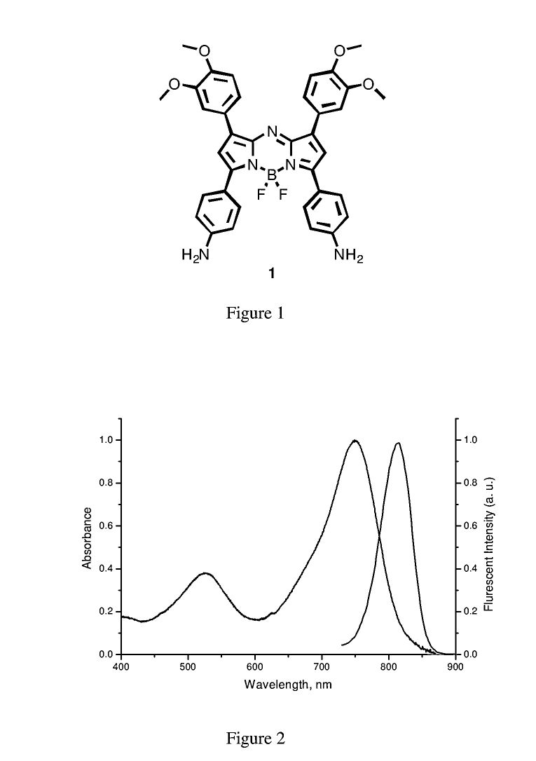

FIG. 2 Normalized absorption and fluorescence spectra of aza-BODIPY compound of the general formula 1 in methanol.

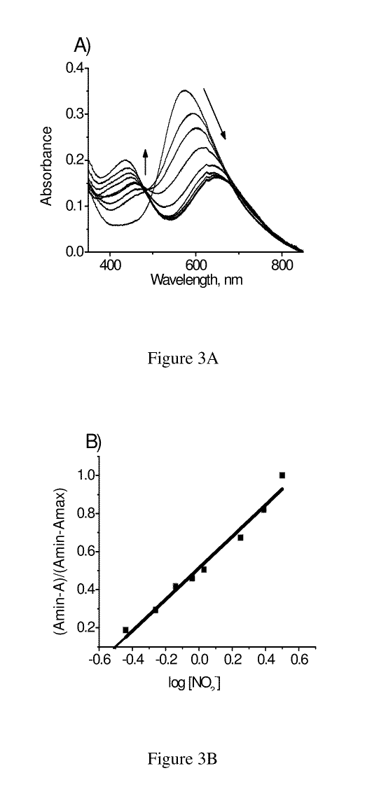

FIG. 3 A) Changes in absorption spectra of the protonated form of the aza-BODIPY dye by successive addition of nitrite ion in water. B) Linear plot for calculation of limit of detection.

FIG. 4 Determination of the unknown concentration of nitrite ions present in a given solution by UV-Vis spectroscopy. A) Absorption spectra of aza-BODIPY compound 1 by addition of different nitrite concentrations from 2.17 μM (0.1 ppm) to 21.7 μM (1 ppm). B) Relative linear plot of absorbance against concentration of nitrite ions; the unknown concentration obtained=15.34 μM (Cᵤₖ=15.37 μM). C) Absorption spectra of the Griess reaction by addition of different nitrite concentrations from 2.17 μM (0.1 ppm) to 21.7 μM (1 ppm) to the Griess reagent. D) Relative straight line plot of the absorbance against concentration of nitrite ions; the unknown concentration obtained=15.0 μM (Cᵤₖ=15.37 μM).

FIG. 5 Bar diagram showing the selectivity of the aza-BODIPY based nitrite sensor towards various competitive anions like SO₄²⁻, Cl⁻, HSO₃⁻, CO₃²⁻, CH₃COO⁻, NO₃⁻, S₂O₃²⁻, N₃⁻ etc. A) Selectivity chart by plotting the decrease in absorbance at 570 nm by addition of different anions. B) Selectivity chart by plotting the bathochromic shift in the absorption maximum of the assay by addition of different anions.

FIG. 6 Photograph of the dipstick device showing its performance in aqueous solution of nitrite ions [2×10⁻⁵ M]; A) The purple colored aza-BODIPY coated over a glass support, B) The dipstick after exposing to the hydrogen chloride vapor, turned to intense blue in color, C) The color change of the dipstick from intense blue to bright green by dipping into a solution containing nitrite ions.

FIG. 7 provides the flow chart showing the synthetic strategy and nitrite sensing.

### DETAILED DESCRIPTION OF THE INVENTION

Accordingly the present invention provides a novel aza-BODIPY compound of formula 1 useful for selective detection of nitrite ions in an aqueous medium.

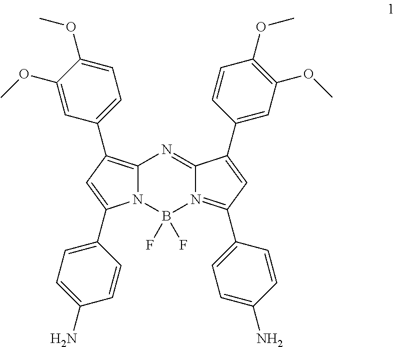

The present invention further provides a process for the preparation of aza-BODIPY compound of formula 1

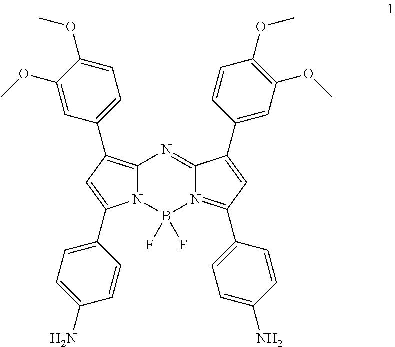

the process comprising:
a) preparing (E)-1-(4-aminophenyl)-3-(3,4-dimethoxyphenyl)prop-2-en-1-one (chalcone) by reacting 4-aminoacetophenone and 3,4-dimethoxybenzaldehyde in ethanol-water (1:1) mixture in the presence of NaOH,
b) preparing 1-(4-aminophenyl)-3-(3,4-dimethoxyphenyl)-4-nitrobutan-1-one by reacting chalcone obtained in step (a) with nitromethane in methanol in presence of activated K₂CO₃,
c) reacting 1-(4-aminophenyl)-3-(3,4-dimethoxyphenyl)-4-nitrobutan-1-one obtained in step (b) with ammonium acetate in distilled ethanol to obtain a reaction mixture, under reflux, for a period of 24 to 36 hours, cooling the reaction mixture to a temperature of 25° C. to 35° C. and removing the ethanol solvent under reduced pressure to obtained a greenish residue, suspending the residue in water and extracting it with dichloromethane, followed by washing the organic layer with brine, drying and concentrating it to obtain the crude product, followed by purification by column chromatography to obtain the desired product (Z)-5-(4-aminophenyl)-N-(5-(4-aminophenyl)-3-(3,4-dimethoxyphenyl)-2H-pyrrol-2-ylidene)-3-(3,4-dimethoxyphenyl)-1H-pyrrol-2-amine (dipyrromethene),
d) reacting the dipyrromethene with borontrifluoride diethyletherate (BF₃.OEt₂) in presence of diisopropylethylamine (DIEA), by using dry dichloromethane as the solvent, under room temperature, stirring for a period of 15-24 hours, washing the above reaction mixture with water and extracting it with dichloromethane, drying and concentrating it to obtain the crude product as purple colored solid, followed by purification through column chromatography in basic alumina using EtOAc-Hexane (1:1) as the eluent to yield the amino aza-BODIPY of formula 1.

In an embodiment of the present invention, the reaction time period used in step (d) is preferably in the range of 15 to 24 hours.

In another embodiment of the present invention, the yield of aza-BODIPY compound of Formula 1 obtained is ca. 65%.

In yet another embodiment, the aza-BODIPY compound of Formula 1 exhibits a highly intense purple color having strong NIR absorption at 750 nm.

In yet another embodiment, the aza-BODIPY compound of Formula 1 exhibits NIR emission in methanol at about 825 nm.

In yet another embodiment, the aza-BODIPY compound of Formula 1 exhibits strong absorption with an extinction coefficient of 4.8×10⁴ M⁻¹cm⁻¹.

In yet another embodiment the aza-BODIPY compound of Formula 1 exhibits high thermal stability up to 280° C.

In yet another embodiment, the aza-BODIPY compound of Formula 1 is useful for the detection of NO₂⁻ ions in solution form.

In yet another embodiment, the aza-BODIPY compound of Formula 1 is useful for the detection of NO₂⁻ ions in solution with a limit of detection of 500 nM.

In yet another embodiment, the aza-BODIPY compound of Formula 1 is useful for the detection of NO₂⁻ ions in presence of other competitive anions.

The present invention further provides a dipstick device useful for selective detection of nitrite ions in water.

The present invention also provides a process for preparing the dipstick device, the process comprising:
i) The aza-BODIPY compound of Formula 1 adsorbed over the ceramic powder silica or alumina; and
ii) said material of step (i) being deposited or fixed over a surface of a strip support.

In an embodiment of the present invention, the strip support used is made up of plastic, glass or paper backing.

In yet another embodiment the dipstick device is useful for detection of nitrite ions in solution.

In still another embodiment, the dipstick device is useful for the testing of analytical sample containing nitrite ion concentration as low as 2×10⁻⁵ M.

The dipstick has particular application in the on the spot detection of nitrite ion containing aqueous samples and the detection event can be conducted at the location where the sample is found and this does not require any sophisticated technical support. The dipstick made up of the aza-BODIPY dye of formula 1 changed its color from purple to intense blue while it is exposed to HCl vapor. The solution containing nitrite ions will result in a distinct color change of the dipstick from intense blue to deep green, where the presence of intense color will indicate high levels of nitrite ions in the sample and a faint color indicate low levels of nitrite in the sample. For the nitrite ions the color of the surface gets changed from bright blue to deep green. All other competitive anions like SO₄²⁻, Cl⁻, HSO₃⁻, CO₃²⁻, CH₃COO⁻, NO₃⁻, S₂O₃²⁻, N₃⁻ will not give any color change to the solid surface. The device can be provided in the form of a kit.

The present assaying is simpler and, does not require sophisticated instruments. The device according to this invention comprises a thermoplastic or glass support strip which measures the presence of nitrite ions in aqueous, clinical and analytical samples by measuring the absorption, wherein it is pre-coated with silica/alumina in order to enable the adsorption of a nitrite selective assay of the Formula 1.

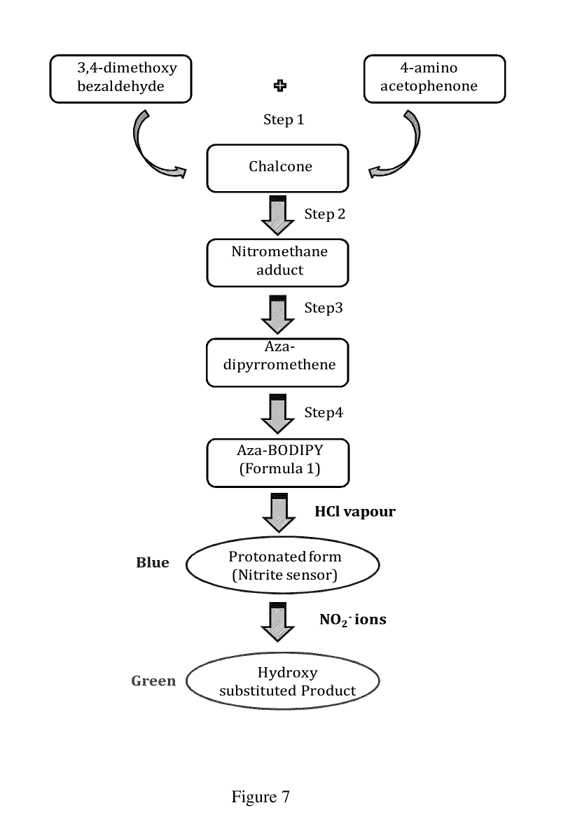

FIG. 7 provides the flow chart showing the synthetic strategy and nitrite sensing.

In the present invention, a novel aza-BODIPY compound of formula 1 has been synthesized. Conversion of the aza-BODIPY to its hydrochloride salt by dissolving it in 1N HCl will make this compound an efficient sensor for detection of nitrite ions in aqueous medium.

To evaluate the amount of nitrite ions in drinking water, biological samples and analytical samples, the present invention comprises of:
1) a novel aza-BODIPY compound having a Formula 1.
2) a practical device called “dipstick device” in which the assay (compound of Formula 1) is conveniently loaded.
3) reaction between nitrite ions and the assay (compound of Formula 1) takes place over the surface of the dipstick and the detection can be signalled by the change in colour over the surface of the dipstick.

The solid surface can be any solid surface, which includes thermoplastic, absorption pads, glass rods and paper strips etc. Preferred supports are thermoplastic and glass, which can be conveniently used by testing personnel, having minimal or no previous experience or training. The preparation and use of such test systems are well described in patents and scientific literatures. Reference may be made to: (a) U.S. Pat. No. 6,406,862 B1 (b) U. S. Pat. No. 20100284858 A1 (c) Y. Takahashi, H. Kasai, H. Nakashini, T. M. Suzuki, Angew. Chem. Int. Ed., 2006, 45, 913. If a stick is used, the assay (formula 1) adsorbed in silica/alumina is bound to one end of the stick such that the end with the assay in silica/alumina can be dipped into sample solution as described for the detection of all ions. The assay is carefully allowed to adsorb over silica/alumina and it is coated over thermoplastic or glass support. Thereby prepared device is ready to use and can dipped into analytical solutions containing nitrite ions. The concentration of nitrite ions as low as 2×10⁻⁵ M and above can be detected.

We have chosen aza-BODIPY compound as the probe for nitrite ions since it shows all the favourable properties of an ideal probe. Preliminary investigations by us indicated that the aza-BODIPY compound of formula 1 shows strong absorption in the NIR region (λₘₐₓ=750 nm) with a high extinction coefficient value of 4.8×10⁴ M⁻¹cm⁻¹.

These dyes exhibited fluorescence emission maximum at 825 nm in methanol. The aza-BODIPY dye of formula 1 has good solubility in common organic solvents and have better photostability. The protonated form of the aza-BODIPY in dilute acid showed a broad blue shifted absorption in the region 570 nm with an intense blue color. The blue color of the protonated species turns to green in the presence of nitrite ion thereby the compound can be used as an efficient nitrite sensor. The color change is due to diazotization reaction between protonated aza-BODIPY and the nitrite ions present in water. The diazonium salts formed by the reaction undergo hydrolysis in aqueous medium to give green hydroxyl substituted product which gets precipitated in the medium.

### EXAMPLES

The following examples are given by way of illustration and therefore should not be construed to limit the scope of present invention.

### Materials and Methods

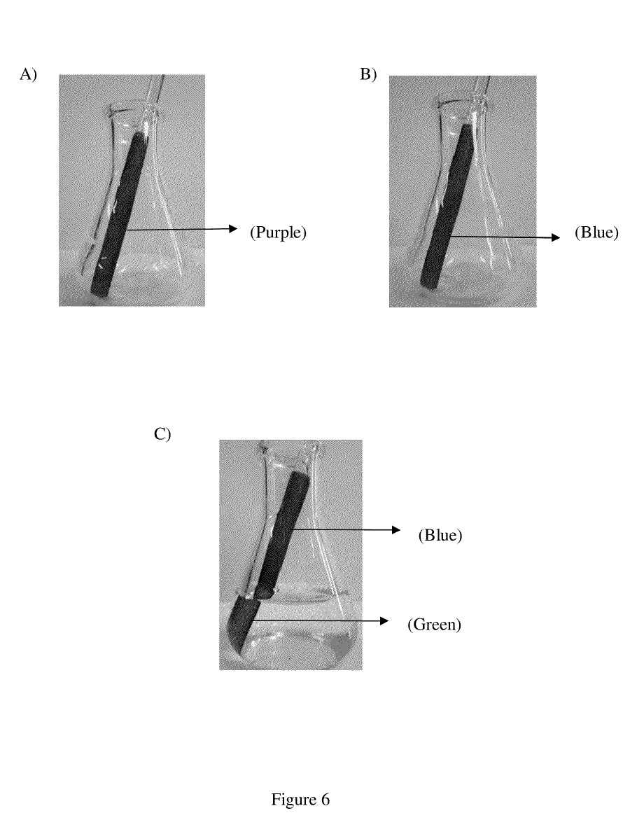

The following MATERIALS and METHODS were used in the examples that follow
1. Thermoplastic or glass supports were cut into small units having 10 cm length and 4 mm radius. This can be prepared in variable length depending upon the nature of the samples to be analyzed.
2. 10 mg of assay 1 (Formula 1) was dissolved in 15 ml dichloromethane. To this solution, 2 g of finely powdered alumina was added and 10 minutes were given for the solvent to evaporate off. The assay containing finely powdered silica/alumina is purple in colour. The fine powdered silica/alumina is bound over the stick up to 5 cm from the bottom of the stick (FIG. 6A). The color of the dipstick will change to intense blue upon exposure of HCl vapor and this can be used for the nitrite detection technique. The change in color from blue to dark green is indication of the presence of nitrite ions. The color change over the surface of the stick can be visually graded or photograph can be taken for permanent record.

Example 1 represents typical synthesis of the aza-BODIPY dye of general formulae 1 and example 2 represents the methodology adopted for the fabrication of dipstick device for the nitrite detection.

Examples 3 and 4 represent the selectivity studies as well as the quantification of nitrite ions.

### Example 1

### Preparation of the aza-BODIPY compound of general formula 1

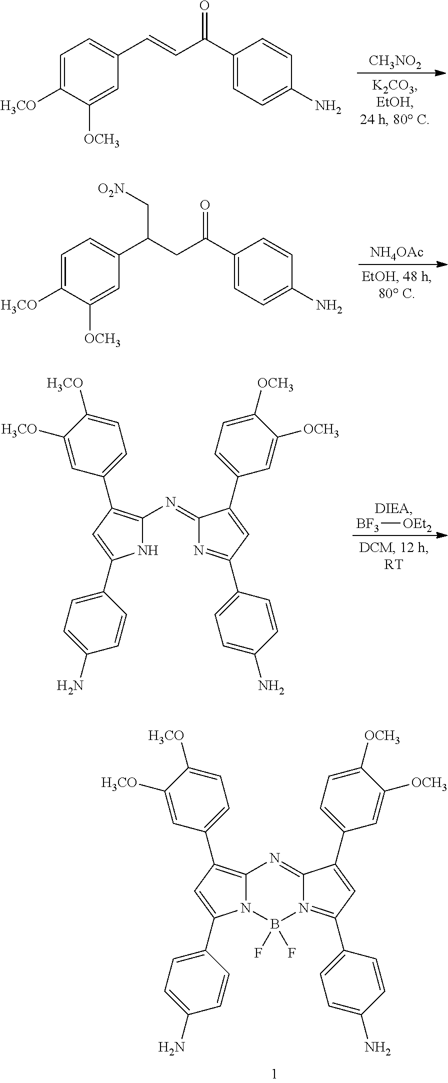

### STEP 1

To the aqueous alcoholic solution of NaOH, 4-aminoacetophenone was added and stirred for 10 min. To this stirred solution 3,4-dimethoxy benzaldehyde was added dropwise and the reaction mixture was stirred at room temperature (25° C.) for 6 h. The precipitated product was filtered under vacuum, washed with ice cold water and dried to give the chalcone (90-95%) as yellow solid. Mp 120-122° C., IR (KBr) vₘₐₓ 3352.28, 1651.07, 1600.92 cm⁻¹. ¹H-NMR (CDCl₃, 500 MHz) δ 7.941 (2H, d, J=8.5 Hz), 7.755 (1H, d, 15.5 Hz), 7.423 (1H, d, J=15.5 Hz), 7.231 (1H, dd), 7.157 (1H, s), 6.901 (1H, d, J=8.5 Hz), 6.712 (2H, d, J=8.5 Hz), 4.168 (2H, s), 3.918 (6H, s). ¹³C NMR (CDCl₃, 125 MHz) δ 188.2, 151.0, 149.2, 143.3, 131.0, 122.8, 120.1, 113.9, 111.2, 110.26, 56.02; FAB-MS m/z Cald for C₁₈H₁₈INO₅ 283.12. Found 283.52.

### STEP 2

The solution of chalcone (5.76 mmol) was dissolved in 80 mL of methanol, activated K₂CO₃ and nitromethane (2 mL) was added to it and refluxed for 24 hours. The mixture was washed with water and extracted with chloroform. The removal of the solvent gave a residue which was separated by column chromatography over silica gel. Elution of the column with a mixture (2:8) of ethyl acetate and hexane gave the product (nitromethane adduct) in good yields. 85%, Mp 138-140° C., IR (KBr) vₘₐₓ 3400.50, 1658.78, 1598.99 cm⁻¹; ¹H-NMR (CDCl₃, 500 MHz) δ 7.778 (2H, d, J=8.5 Hz), 6.807 (2H, s), 6.771 (1H, s), 6.634 (2H, d, J=8.5 Hz), 4.838 (1H, q), 4.650 (1H, q), 4.180 (2H, s), 4.156 (1H, t), 3.862 (3H, s), 3.841 (3H, s), 3.363 (2H, m); ¹³C NMR (CDCl₃, 125 MHz) δ 194.9, 151.6, 149.3, 148.6, 132.4, 130.6, 127.0, 119.3, 113.8, 111.7, 111.2, 79.9, 56.1, 41.1, 39.4; FAB-MS m/z Cald for C₁₈H₁₈INO₅ 344.14. Found 344.75.

### STEP 3

The nitromethane adduct (2.45 mmol) and ammonium acetate (7.6 g, 95 mmol) were dissolved in ethanol (20 mL) and heated under reflux for 48 h. The precipitated product was filtered, washed with cold ethanol, dried and recrystallized from chloroform to give the azadipyrromethene as greenish crystals with a metallic luster. 40%, Mp 170-172° C.; IR (KBr) vₘₐₓ 3361.93, 1600.92, 1506.41 cm⁻¹; ¹H-NMR (CDCl₃, 300 MHz) δ 7.79 (4H, d, J=8 Hz), 7.61 (2H, d, J=8 Hz), 7.53 (2H, s), 6.99 (2H, s), 6.92 (2H, d, J=8 Hz), 6.81 (4H, d, J=8.5 Hz), 4.06 (4H, s), 3.93 (6H, s), 3.74 (6H, s); ¹³C NMR (CDCl₃, 75 MHz) δ 153.9, 149.2, 148.9, 148.8, 148.3, 127.5, 121.8, 115.2, 113.2, 112.4, 111.0, 56.0, 55.8; FAB-MS m/z Cald for C₃₆H₃₁N₃O₄ 599.23. Found 599.26.

### STEP 4

The azadipyrromethene (0.45 mmol) dissolved in dry dichloromethane (80 mL) was treated with DIEA (diisopropylethylamine) (0.8 mL, 4.6 mmol) and stirred for 10 min at 30° C. To this reaction mixture, boron trifluoride diethyl etherate (1 mL, 8.13 mmol) was added and stirred at room temperature (25° C.) for 10 h. The solvent was evaporated, washed with water (2×50 mL) and extracted with chloroform. Removal of the solvent gave a residue, which was separated by column chromatography over basic alumina. Elution of the column with a mixture (1:1) ethyl acetate and hexane gave the aza-BODIPY of formula 1 as purple colored solid. 75%, Mp 192-194° C.; IR (KBr) vₘₐₓ 3360.00, 1600.92, 1498.69 cm⁻¹; ¹H-NMR (CDCl₃, 500 MHz) δ 8.00 (4H, d, J=8.5 Hz), 7.64 (2H, dd, J=8 Hz), 7.55 (2H, d, J=2 Hz), 6.94 (4H, d, J=6.5 Hz), 6.75 (4H, d, J=8.5 Hz), 4.09 (4H, s), 3.95 (6H, s), 3.81 (6H, s); ¹³C NMR (CDCl₃, 125 MHz) δ 156.3, 154.2, 150.8, 149.2, 137.1, 136.9, 130.9, 130.8, 130.3, 129.5, 125.7, 122.9, 122.8, 117.6, 112.2, 111.2, 56.1, 55.9; FAB-MS m/z Cald for C₃₆H₃₀BF₂N₃O₄ 647.25. Found 647.46.

### Example 2

### Preparation of Dipstick for the on-Site Analysis of Nitrite Ions

10 mg of compound 1 (Formula 1) was dissolved in 15 ml dichloromethane, to this solution, 2 gm of finely powdered alumina was added and 10 minutes were given for the solvent to evaporateoff. The assay containing finely powdered silica/alumina was purple in colour. The fine powdered silica/alumina was bound over the stick up to 5 cm from the bottom of the stick (FIG. 6A). The color of the dipstick will change to intense blue upon exposure of HCl vapor (FIG. 6B) and this can be used for nitrite detection technique. The change in color from blue to dark green is an indication of the presence of nitrite (FIG. 6C). The color changes over the surface of the stick can be visually graded or photograph can be taken for permanent record.

Thermoplastic or glass support was cut as 10 cm long and having 4 mm radius. Assay containing silica/alumina was carefully fixed over the surface of the support and said dipstick was ready to use. The stick was exposed to HCl gas followed by dipping into a solution taken in a beaker containing nitrite ions (2×10⁻⁵ M). Wherever the analyte will get in contact with the stick, a color change will be observed from deep blue to intense green, which indicates the presence of nitrite ions.

### Example 3

### Selectivity studies with competitive anions

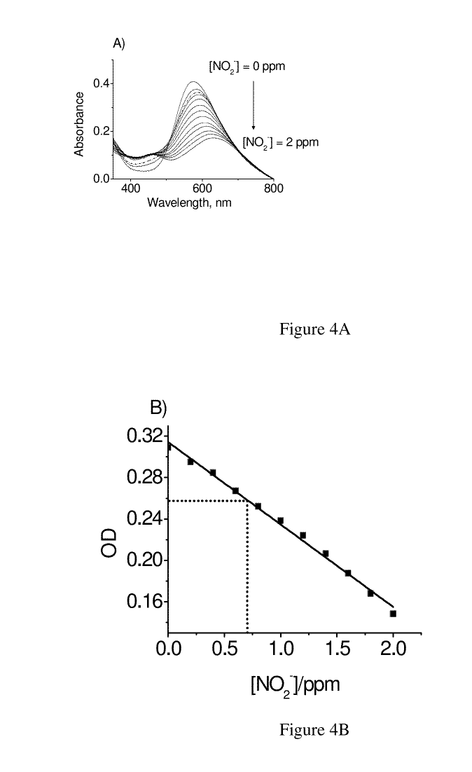

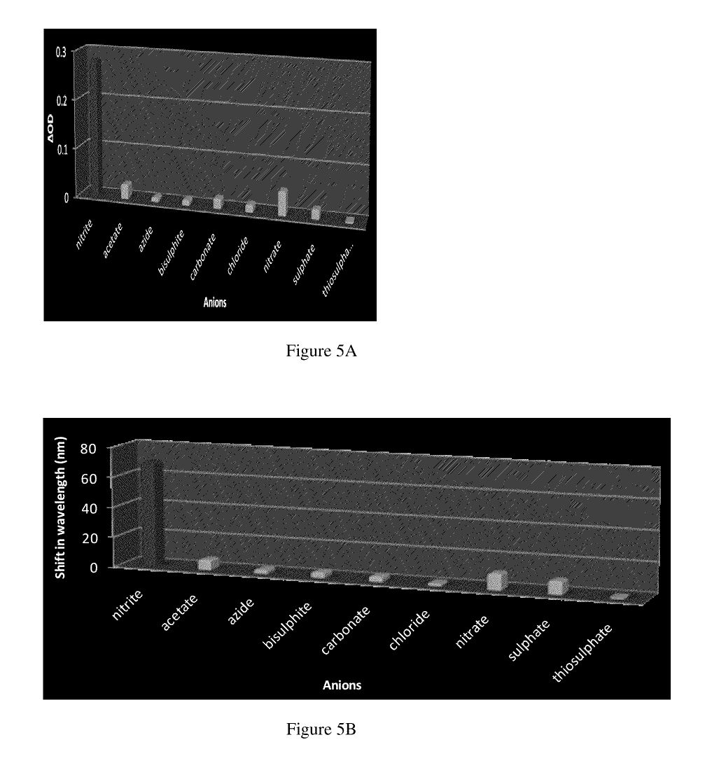

The stock solution (2 μM) of the aza-BODIPY compound of formula 1 was prepared in 1N HCl and the solutions of different competitive anions such as SO₄²⁻, Cl⁻, HSO₃, CO₃²⁻, CH₃COO⁻, NO₃⁻, S₂O₃²⁻, N₃⁻ and NO₂⁻ ions were prepared in water. We have performed the titration experiments by gradual addition of various anions in water to the aza-BODIPY of formula 1. Only nitrite ion induces a hypochromicity of 70% and a bathochromic shift of 60 nm (FIG. 3) to the protonated form of the aza-BODIPY compound of formula 1 whereas the other competitive anions showed negligible changes in the absorption even at 100 fold higher concentrations compared to nitrite ions (FIG. 5). For example, the addition of nitrate ions to the assay solution does not make any significant changes in the absorption spectra and also to the colour of the solution.

### Example 4

### Quantification of Nitrite Ions in Water

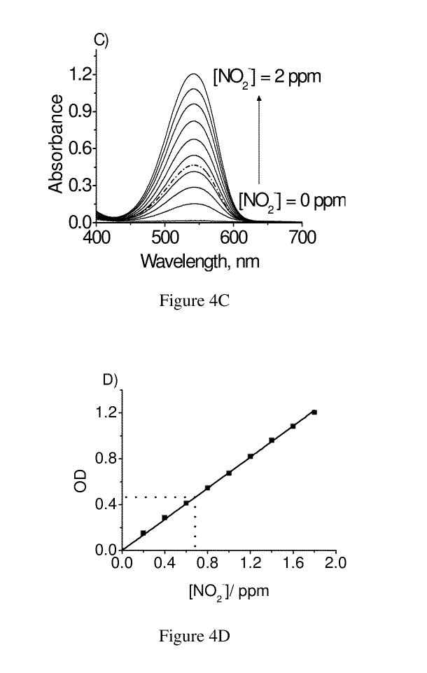

To investigate the potential of the aza-BODIPY compound of formula 1 in the determination of nitrite concentration, the probe was treated with nitrite ions at various concentrations (0 to 2 ppm). The final concentration of probe 1 was maintained at 0.1 ppm, while the concentrations of nitrite ion were varied from 0 to 2 ppm. The concentration of nitrite ions versus absorbance at 570 nm follows linearity. The point at which the absorbance of the unknown sample meets the straight line will give the amount of nitrite ions present in the sample solution (FIG. 4). We compared the efficacy of the aza-BODIPY of formula 1 with the commercial variant Griess reagent in the similar manner and we observed that the aza-BODIPY compound of formula 1 gave accurate results in the quantification of nitrite ions. The aza-BODIPY compound of formula 1 can detect the nitrite ions in water with a limit of detection (LOD) of 500 nm (5×10⁻⁷ M) whereas in the case of dipstick strategy, the LOD is 20 μM (2×10⁻⁵ M).

### Advantages

The aza-BODIPY dye used for the present invention possesses satisfactory properties of an ideal sensor and can be used as a simple, efficient and economical sensor for nitrite ions. The main advantages of the present invention are:

1. The aza-BODIPY compound of formula 1 is novel single molecule.

2. The synthetic methodology adopted for the preparation of aza-BODIPY dye of formula 1 is commercially viable.

3. The aza-BODIPY compound of formula 1 possesses absorption in the near-infrared region (500-750 nm).

4. The aza-BODIPY compound of formula 1 possesses fluorescence emission in the near-infrared region (750-850 nm).

5. The aza-BODIPY compound of formula 1 showed thermal stability up to 280° C.

6. The aza-BODIPY compound of formula 1 can be used for the detection of nitrite ions in aqueous medium.

7. The aza-BODIPY compound of formula 1 showed a limit of detection (LOD) of 500 nM (5×10⁻⁷ M) in the detection of nitrite ions in aqueous medium.

8. The excellent selectivity shown by the assay with formula 1 towards nitrite ions among other important competitive ions like SO₄²⁻, Cl⁻, HSO₃⁻, CO₃²⁻, CH₃COO⁻, NO₃⁻, S₂O₃²⁻, N₃⁻ etc.

9. The aza-BODIPY compound can be used to quantify the amount of nitrite ions in solutions by spectroscopic techniques like UV-spectrophotometer.

10. In addition to this, the compound having formula 1 can be more conveniently used as a device by coating the assay in silica/alumina over a thermoplastic or a glass solid support, which can be used as dip stick.

11. The present invention makes the fabrication of present device so comfortable that it can be handled and used very easily and does not require any expertise or sophisticated instruments.

12. The lowest concentration of 2×10⁻⁵ M can be detected using the dipstick technique.

1-17. (canceled)

18. An aza-BODIPY compound of formula 1 and salts thereof

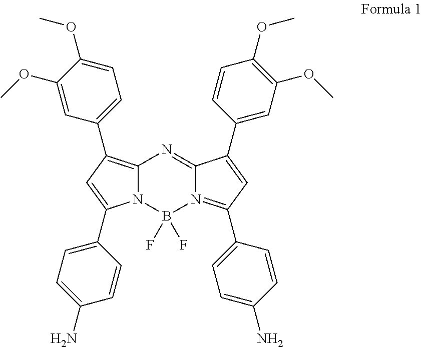

19. A process for the preparation of the aza-BODIPY compound of claim 18, said process comprising:
a) adding 4-aminoacetophenone to a solution of NaOH and stirring for 10 minutes to obtain a reaction mixture;
b) adding 3,4-dimethoxy benzaldehyde dropwise to the reaction mixture and stirring for 6 hours to obtain a precipitate;
c) filtering said precipitate and washing with water to obtain a chalcone;
d) reacting the chalcone in methanol with nitromethane in presence of activated K₂CO₃ and refluxing for a time period of 24 hours to obtain a mixture;
e) washing the mixture with water and extracting with chloroform;
removing the solvent from the mixture of step (e) to obtain a residue and separating the residue by column chromatography to obtain a nitromethane adduct;
g) reacting the nitromethane adduct with ammonium acetate in ethanol and heating under reflux for a time period of 48 hours to obtain a product;
h) filtering the product and washing with ethanol, drying and recrystallizing from chloroform to obtain azadipyrromethene as greenish crystals with a metallic luster;
i) reacting the azadipyrromethene in dry dichloromethane with boron trifluoride diethyl etherate in presence of diisopropylethylamine (DIEA) and stirring for a time period of 15 hours at a temperature of 25° C.;
j) evaporating the solvent to obtain a residue and washing the residue with water followed by extraction with chloroform; and
k) separating the washed residue by column chromatography to obtain the aza-BODIPY compound as a purple colored solid.

20. A dipstick device for use in selective detection of NO₂⁻ ions in water, wherein the device comprises the compound of claim 18 fixed on the surface of a support selected from the group consisting of plastic, glass and paper backing.

21. A process for preparing the dipstick device as claimed in claim 20, said process comprising the steps:
a. dissolving said compound in dichloromethane to obtain a mixture;
b. adding finely powdered silica or alumina to the mixture obtained in step (a) and allowing the dichloromethane to evaporate after leaving for 10 minutes to obtain an assay; and
c. fixing the assay obtained in step (b) over the surface of a support to obtain the dipstick device.

22. The process as claimed in claim 21, wherein the support is made of plastic, glass or paper backing selected from the group consisting of thermoplastic, absorption pads, glass rods and paper strips.

23. A method for selective detection of NO₂⁻ ions in a sample, comprising:
(i) exposing the dipstick device of claim 20 to HCl gas, followed by;
(ii) dipping said dipstick device in said sample,
wherein a change of color of said dipstick device from deep blue to intense green indicates the presence of NO₂⁻ ions.

24. The process as claimed in claim 23, wherein the sample is selected from the group consisting of an aqueous sample, a clinical sample and an analytical sample.

25. The dipstick device as claimed in claim 20, wherein said device is able to detect nitrite ion in an analytical sample at a concentration as low as 2.0±0.5×10⁻⁵ M.
